(19) United States
(12) Patent Application Publication
(10) Pub. No.: US 2004/0147938 A1
(43) Pub. Date: Jul. 29, 2004

(54) System for IOL insertion

(75) Inventors: Vaclav Dusek, Bellevue, WA (US); Robert Ziegler, Meridian, ID (US)

Correspondence Address: STRADLING YOCCO CARLSON & RAUTH, SUITE 1600, 660 NEWPORT CENTER DRIVE, P.O. BOX 7680, NEWPORT BEACH, CA, 92660, US

(73) Assignees: MicroSurgical Technology; Advanced Medical Optics

(21) Appl. No.: 10/672,275

(22) Filed: Sep. 24, 2003

Related U.S. Application Data
(60) Provisional application No. 60/413,512, filed on Sep. 25, 2002.

(51) Int. Cl. A61F 9/00

(52) U.S. Cl. 606/107

(57) ABSTRACT

An apparatus for controllably delivering an IOL into the eye of a patient is disclosed. The device is configured for convenient and reliable positioning of a plunger rod assembly at different stages of the IOL injection procedure, such that various aspects of the insertion procedure can be performed precisely and reliably by a user of the device. The present invention may include a latch pin and a latch finger configured for engagement with the latch pin to prevent proximal lateral movement of the plunger rod assembly so that the intra-ocular lens may be accurately positioned within the eye. The device assists the user during the procedure by accurately and consistently positioning the plunger rod assembly prior to ejection of the folded or rolled IOL from the insertion device.

### CROSS REFERENCE TO RELATED APPLICATIONS

The present application claims priority to U.S. Provisional Patent Application No. 60/413,512, filed Sep. 25, 2002, entitled “SYSTEM FOR IOL INSERTION,” the contents of which are fully incorporated herein by reference.

### BACKGROUND OF THE INVENTION

This invention pertains to eye surgery in which an artificial intra-ocular lens (IOL) is inserted through the cornea into a lens capsule.

When a natural lens becomes occluded, it is conventional practice to remove the occluded lens, such as by phaco emulsification, and to replace the natural lens with an artificial intra-ocular lens (IOL). In order to decrease the size of the corneal incision required for insertion of the IOL, IOLs can be formed of resilient material that can be “folded” or rolled for insertion into the lens capsule. Various types of devices have been proposed for folding, holding, and injecting such IOLs. Examples are shown in U.S. Pat. No. 4,681,102 which has been cited in a large number of later issued patents including U.S. Pat. Nos. 6,334,862 and 6,398,788. Each of these patents shows a cartridge into which an IOL can be inserted, followed by folding or rolling of the IOL by manipulation of components of the cartridge. The cartridge is attached to or otherwise held in an insertion device or “shooter” which can include a plunger or push rod that is forced through the cartridge. Typically the cartridge includes a distal ejection tube which is inserted into a small corneal incision and through which the folded or rolled IOL is ejected by forcing the push rod through the cartridge.

The present invention provides a system for convenient and reliable positioning of the rod at different stages of the IOL injection procedure, such that aspects can be performed precisely and reliably by a skilled technician rather than the surgeon. In addition, the present invention assists the surgeon during the procedure by accurately and consistently positioning the push rod, prior to ejection of the folded or rolled IOL from the insertion instrument.

### BRIEF DESCRIPTION OF THE DRAWINGS

Other features and advantages of the present invention will be seen as the following description of particular embodiments progresses in conjunction with the drawings, in which:

FIG. 1 is perspective view of an embodiment of an insertion device in accordance with the present invention;

FIG. 2 is an exploded view of an embodiment of an insertion device in accordance with the present invention;

FIG. 3 is a perspective view of an embodiment of an intra-ocular lens holding cartridge in accordance with the present invention;

FIG. 4 is a perspective view of an embodiment of a cylindrical barrel in accordance with the present invention;

FIG. 5 is a top plan view of an embodiment of a cylindrical barrel in accordance with the present invention;

FIG. 6 is side sectional view of an embodiment of a cylindrical barrel in accordance with the present invention;

FIG. 7 is a front end elevation view of an embodiment of a cylindrical barrel in accordance with the present invention;

FIG. 8 is a rear end elevation view of an embodiment of a cylindrical barrel in accordance with the present invention;

FIG. 9 is a perspective view of an embodiment of a clip in accordance with the present invention;

FIG. 10 is a top sectional view of an embodiment of a clip in accordance with the present invention;

FIG. 11 is a side sectional view of an embodiment of a clip in accordance with the present invention;

FIG. 12 is an end view of an embodiment of a clip in accordance with the present invention;

FIG. 13 is a sectional view of a distal end of an embodiment of a clip in accordance with the present invention;

FIG. 14 is a perspective view of an embodiment of a pushing member in accordance with the present invention;

FIG. 15 is a side perspective view of an embodiment of a pushing member in accordance with the present invention;

FIG. 16 is an end view of an embodiment of a pushing member in accordance with the present invention;

FIG. 17 is a perspective view of an embodiment of an elongate body in accordance with the present invention;

FIG. 18 is a side perspective view of an embodiment of an elongate body in accordance with the present invention;

FIG. 19 is a sectional view of an embodiment of an elongate body in accordance with the present invention;

FIG. 20 is an end view of an embodiment of an elongate body in accordance with the present invention;

FIG. 21 is a sectional view of a distal end of an embodiment of an elongate body in accordance with the present invention;

FIG. 22 is a perspective view of an embodiment of a latch pin in accordance with the present invention;

FIG. 23 is an end view of an embodiment of a latch pin in accordance with the present invention;

FIG. 24 is a side view of an embodiment of a latch pin in accordance with the present invention;

FIG. 25 is a perspective view of an embodiment of a control knob assembly in accordance with the present invention;

FIG. 26 is an end view of an embodiment of a control knob assembly in accordance with the present invention;

FIG. 27 is a sectional view of an embodiment of a control knob assembly in accordance with the present invention; and

FIGS. 28-32 illustrate various stages of a method of operating an embodiment of an insertion device in accordance with the present invention.

### DETAILED DESCRIPTION OF THE INVENTION

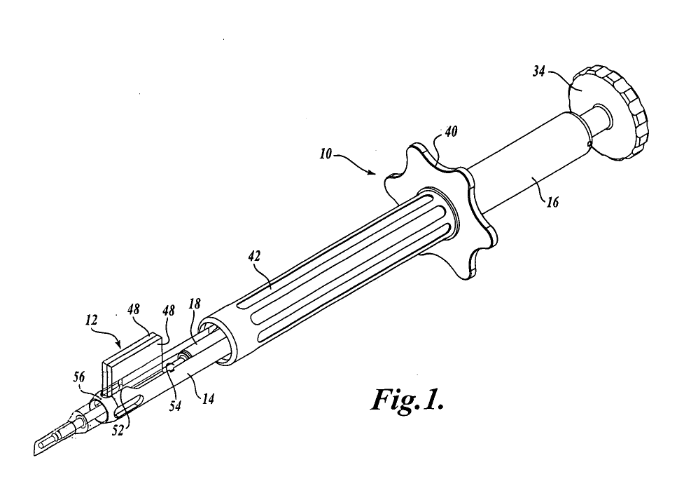

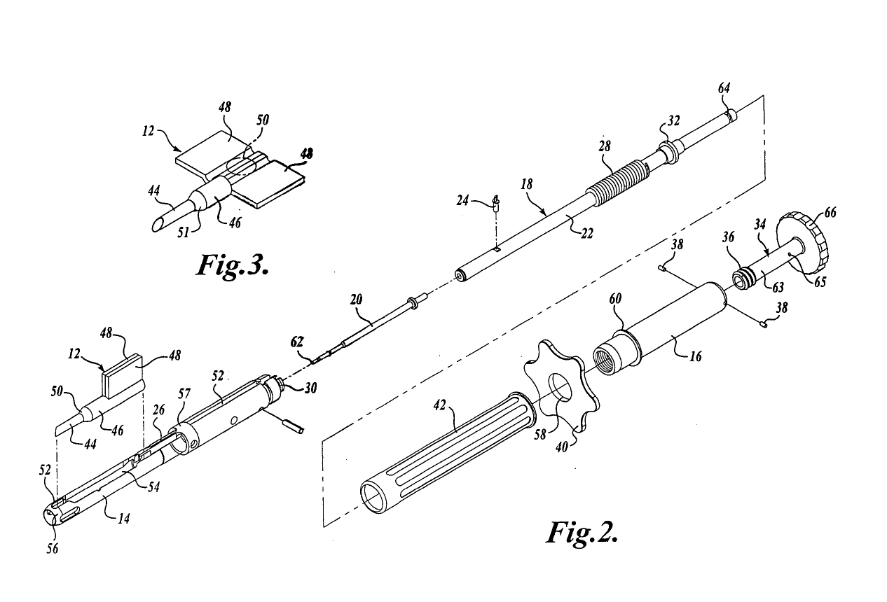

An embodiment of the instrument 10 of the present invention is shown in FIGS. 1-2, FIG. 1 illustrating the parts in assembled relationship, and FIG. 2 showing most parts in exploded relationship. FIG. 3 shows a conventional lens-holding cartridge 12 apart from the remainder of the instrument.

With reference primarily to FIG. 2, as described in more detail below, the lens holding cartridge 12 can be fitted in a distal portion 14 of an elongated, generally cylindrical barrel. The barrel has a proximate portion 16 secured to the distal portion 14. A push rod 18 is slideable fore and aft (distally and proximally) in the bore of the barrel. Push rod 18 has a distal part 20 aligned with and secured to a proximate part 22. A transversely projecting latch pin 24 is carried by the proximate part 22. The latch pin cooperates with a latch finger 26 carried on the barrel proximate part 14.

A helical compression spring 28 is slideable along the rod proximate part 22 and acts between the proximate end 30 of the barrel part 14 and a slide disc 32 carried on the rod proximate part 22. Rearward travel of the slide is limited by engagement against an inturned lip of the barrel distal part 16 and/or one or more transverse pins 38 at the proximate end portion. An operating handle or knob 34 is rotatably secured to the proximate end portion of the push rod part 22. In addition, the operating knob has coarse external threads 36 at its distal end portion which cooperate with pins 38 that project inward from the proximate end portion of the barrel part 16.

Preferably the instrument also includes a transversely projecting finger support 40 secured to the barrel part 16 at a convenient distance from its proximate end, and a protective sheath 42 spaced outward from and extending over a substantial segment of the barrel, including the portion having the latch finger 26.

As described in more detail below, the knob 34 is manipulated to control the position of the push rod 18 which extends into the lens-holding capsule 12, and in a controlled fashion, first positions the folded or rolled IOL for projection from the cartridge, then can be used to force the IOL farther through the cartridge and hold it in a partially projected position before final injection of the IOL into the lens capsule.

Lens Holding Cartridge

With reference to FIG. 3, cartridge 12 is of the same general design as the cartridge shown in U.S. Pat. No. 6,398,788 and U.S. Pat. No. 6,334,862. The distal end portion of the cartridge is a hollow ejection tube 44, leading to a larger diameter holding chamber 46. Lens-folding wings or tabs 48 can be formed integrally with tube 44 and chamber 46 and are movable relative to each other by means of an integral hinge joint. With the tabs 48 folded “open” as shown in FIG. 3, an IOL 50 can be manually positioned centrally between the tabs as is conventional, followed by swinging the tabs together to fold or roll the lens. In the “closed” position (FIGS. 1 and 2) the tabs 48 project radially beyond the loading chamber 46 of the cartridge, and the folded or rolled IOL is aligned with the larger bore of the chamber 46. A tapered connecting portion 51 leads from the chamber 46 to the ejection tube 44.

Barrel

The distal portion 14 of the barrel has a long slot 52 extending in an axial direction. This slot communicates between the interior bore and the exterior of the barrel. A wider portion 54 of the slot allows the loaded cartridge 12 to be inserted downward into the barrel bore and slid forward (distally) to the position shown in the FIG. 1 in which the tabs 48 are held closed by engagement in the narrower distal portion of the slot 52. Forward movement of the cartridge is limited by the continuous distal ring 56 of the barrel. The latch finger 26 is centered over the slot 52, generally centrally of the barrel portion 14. Finger 26 is cantilevered from a mounting ring 57 secured on barrel part 14.

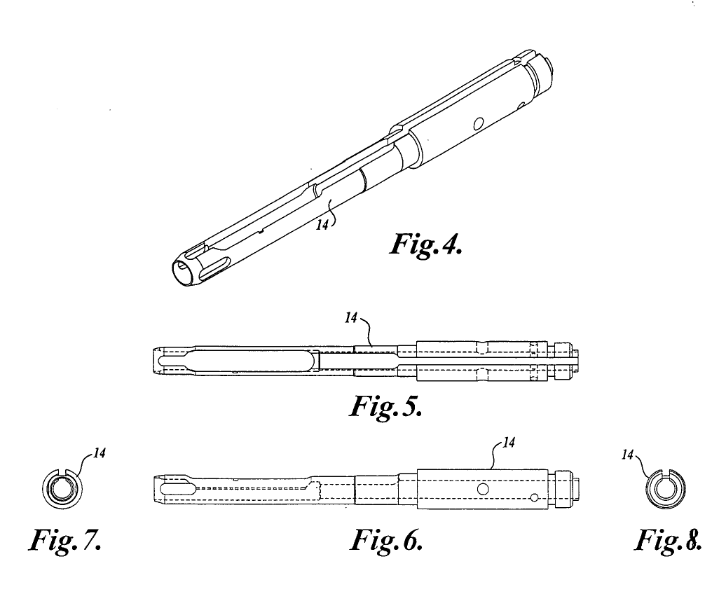

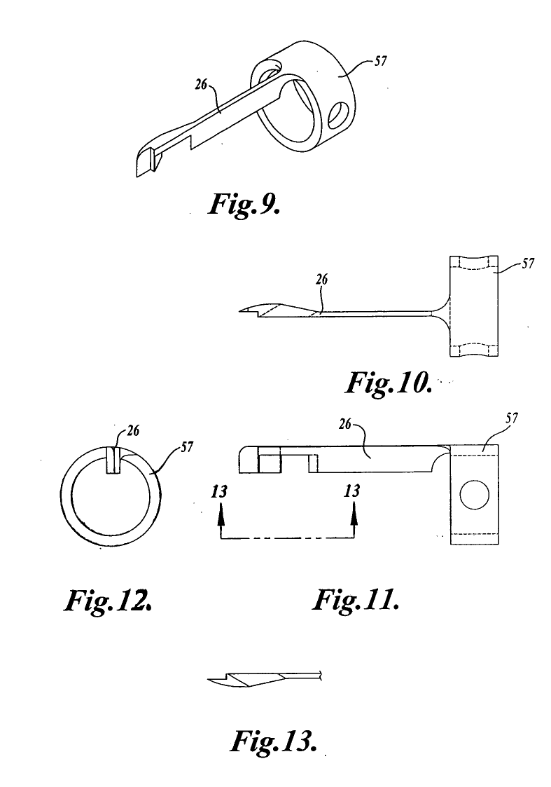

The distal portion 14 of the barrel is shown in greater detail in FIG. 4 (top perspective), FIG. 5 (top plan), FIG. 6 (side elevation), FIG. 7 (front end elevation), and FIG. 8 (rear end elevation). The latch finger 26 and its mounting ring 57 are shown in more detail in FIG. 9 (top perspective), FIG. 10 (top plan), FIG. 11 (side elevation), FIG. 12 (front end elevation), and FIG. 13 (fragmentary bottom plan).

Returning to FIG. 2, the proximate barrel portion 16 carries the finger support 40. For example, the finger support can have a central aperture 58 sized for fitting over the distal end portion of barrel part 16, such as against an annular rib or shoulder 60. The finger support can be press-fitted on the barrel portion 16 or secured in any other convenient manner. Similarly, the protective sheath 42 is mounted to the finger support 40 or part 16 and projects distally therefrom. Barrel parts 14 and 16 are secured together end to end. In the illustrated embodiment, the distal end 30 of part 14 is formed with an externally threaded stem. The hollow interior of the proximate barrel part 16 is aligned axially with the bore of the distal barrel part 14 and has internal threads to mate with the threads of the stem 30. The manner of connection of the two (14, 16) is not important. For example, either part can be partially telescoped within the other and secured by pins, press fit, or any other effective and convenient manner. The sheath 42 extends from the proximate barrel part 16 over the distal barrel part 14 so as to cover the latch finger 26.

Push Rod Assembly

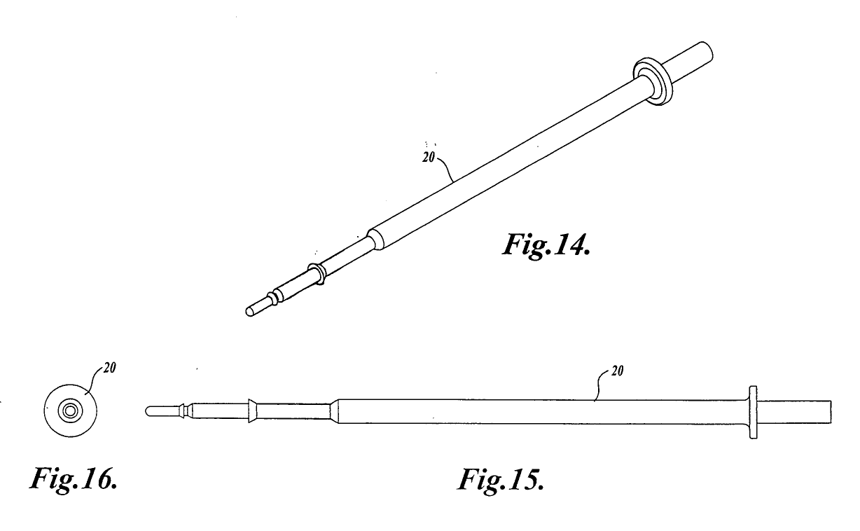

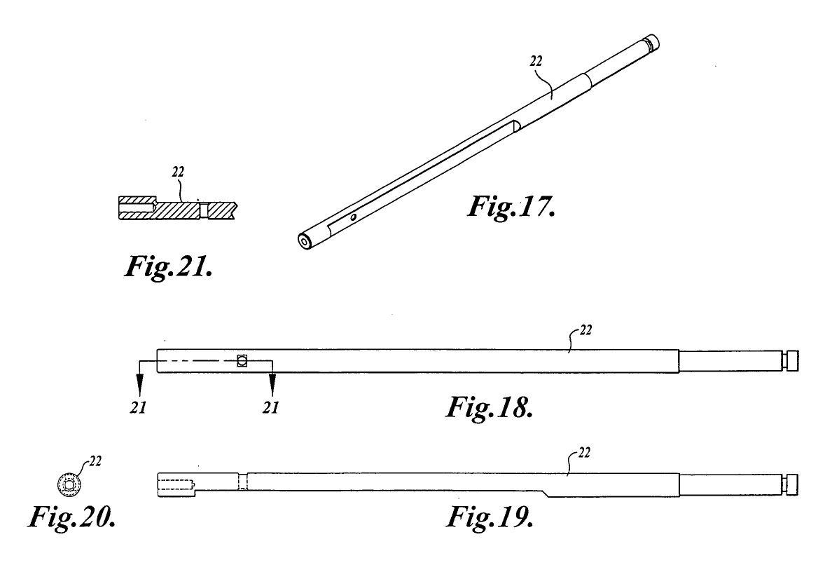

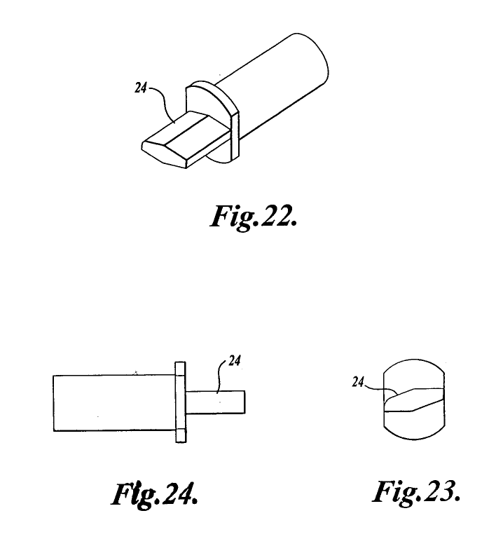

In general, the push rod 18 slides fore and aft in the barrel by manipulation of the operating handle or knob 34. The distal rod part 20 (see also FIGS. 14-16) has a distal end 62 positioned to slide into the bore of the lens holding cartridge 12. This part is secured to the proximate part 22 (see also FIGS. 16-21 and note that different parts are drawn to different scales; for example, the proximate end portion of part 20 shown in FIGS. 14-16 fits tightly in a blind socket in the distal end of part 22 of FIGS. 17-21). The proximate part 22 and corresponding sections of the barrel are configured to allow the fore and aft sliding without appreciable relative rotation. This is to reliably maintain the latch finger 24 (shown greatly enlarged in FIGS. 22-24) aligned with the barrel slot 52 and the latch finger 26. The stem of the latch finger can be press fitted in a transversely extending bore of the distal push rod part 22. The helical compression spring 28 fits over the proximate portion of push rod part 22. Similarly, slide 32 is moveable along the proximate end portion of part 22. The slide has an annular rim or shoulder against which the proximate end of the spring 28 acts. The other, distal end of the spring acts against the proximate end of the treaded stem 30. The effect is to force the slide rearward (proximally) along the push rod, but the travel of the slide is limited by engagement against the pins 38 that project inward from the proximate end of the barrel portion 16.

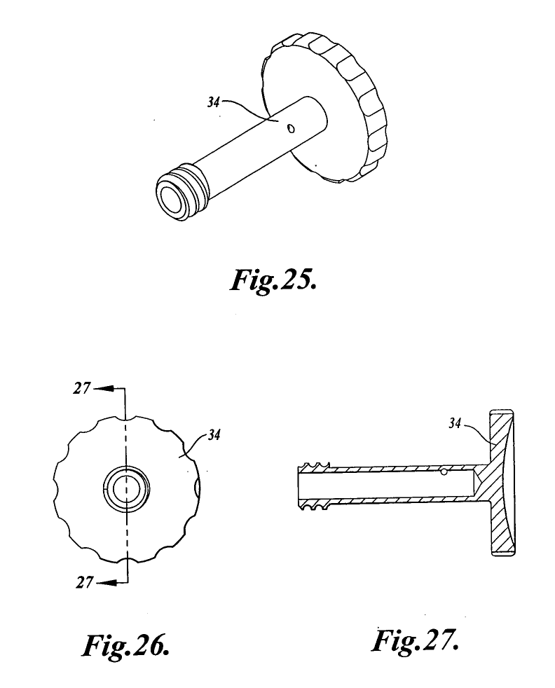

The operating knob 34 (FIGS. 25-27) is rotatably connected to the proximate end portion of push rod part 22. More specifically, the operating knob has a distally extending hub 63 with an axial bore fitted over the proximate end portion of push rod part 22. Part 22 has an annular groove 64 aligned with a pin 65 extending inward into the bore of the hub 63. Fore and aft movement of the operating knob, achieved conveniently by use of the larger diameter knurled end disc 66, results in equivalent fore and aft sliding movement of the push rod 18. However the operating knob is free to rotate relative to the push rod without corresponding rotation of the rod.

The distal end portion of the operating handle hub has the external threads 36 which cooperate with the inward projecting pins 38 of barrel part 16 for a threaded fit of the operating knob with the barrel part 16 over a limited distance.

Operation

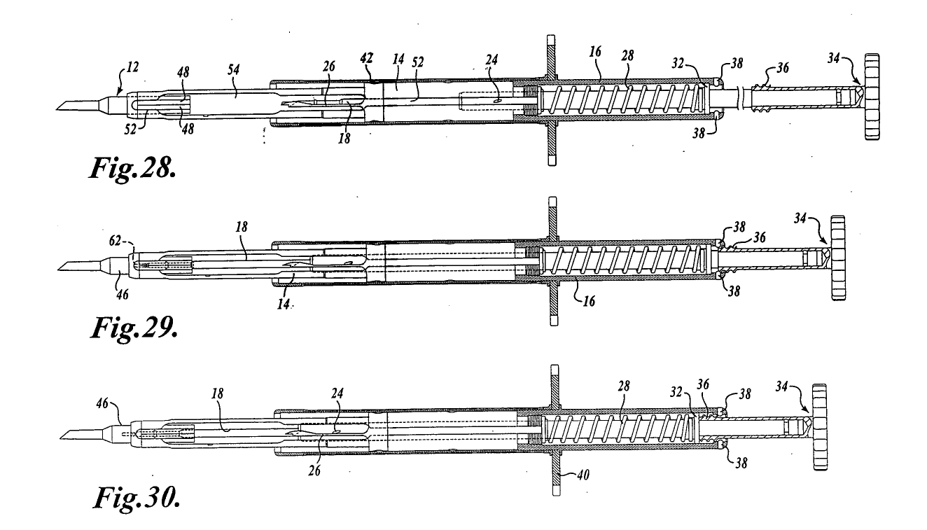

With reference to FIGS. 28-32, and starting with FIG. 28, the instrument in accordance with the present invention is prepared for use by first fitting an IOL in the lens-holding cartridge 12, folding or rolling the IOL by manipulation of the cartridge tabs 48, insertion of the cartridge downward through the wider barrel slot portion 54, and forward (distal) shifting of the cartridge such that the tabs 48 fit in the narrower distal part 52 of the barrel slot. During this procedure, the push rod 18 is retracted rearward from the wider slot portion 54. Note that the latch pin 24 is positioned far to the rear (proximally) of the latch finger 26; the external threads 36 of the operating knob 34 are disengaged from the pins 28; and the compression spring 28 forces the slide 32 rearward (proximally) adjacent to the proximate end of the barrel part 16.

With reference to FIG. 29, the operating knob 34 then is slid forward (distally) to move the push rod 18 distally relative to the composite barrel 14, 16. Threads 36 butt against pins 38 at a position in which the distal end 62 of the push rod is approximately aligned with the distal end of the lens-folding tabs, i.e., the lens is forced from between the tabs into the holding chamber 46. Pins 38 block additional linear sliding movement of the push rod in the barrel.

In order to advance the push rod farther, it is necessary to rotate the operating knob 34. The external threads 36 cooperate with the pins 38 such that the knob may be turned in a direction to gradually advance the push rod from the position of FIG. 29 to the position of FIG. 30. At the same time, the distal end of the operating knob is engaged against the slide 32, and the slide 32 moves distally against the force of the compression spring 28. The axial extent of the threads 36 is limited. After a relatively short travel, sufficient to move the distal end of the push rod approximately half way into the holding chamber 46, the proximate end of the externally threaded portion 36 clears the pins 38 and no additional advancement of the push rod occurs by continuing to rotate the operating knob 34. Rather, the user, typically a technician, will know that this position (FIG. 30) has been reached because the end thread will “click” over the pins 38 as the slide and operating knob are biased rearward (proximally) by the compression spring 28.

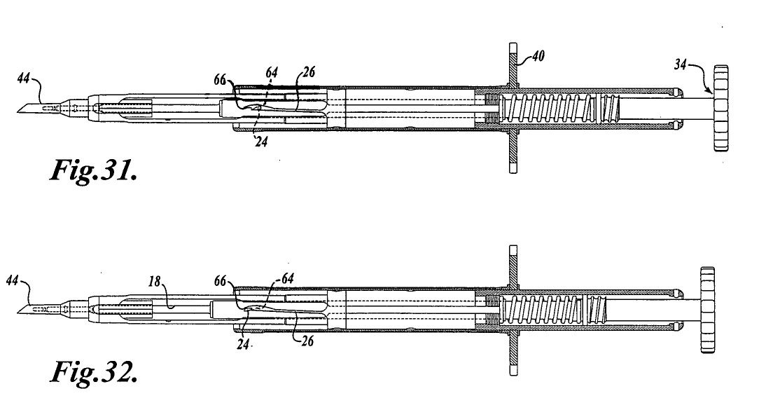

In the position of FIG. 30, the latch pin 24 is positioned generally alongside the center portion of the latch finger 26. Further advancement of the push rod and latch pin are achieved by pressing the operating knob 34 inward relative to the barrel of the instrument, such as by engagement of the knob with the thumb or palm while the fingers hook against the finger support 40. The distal end of the push rod forces the IOL from the holding chamber 46 and farther into the ejection tube 44, as seen in FIG. 31. As this occurs, the leading end of the latch pins engages an angled segment or ramp 64 of the pin, wedging the pin sideways, upward in the orientation of FIG. 31. The upward movement of the latch finger is contrary to its natural, centering resiliency. Once the latch pin has cleared a notched segment 66 at the distal end of the latch finger, the latch finger snaps back toward the central, relaxed position. The inward directed force on the operating knob applied by the user's hand is relieved, and the proximate end of the latch pin 24 will engage in the notch, preventing retraction of the push rod 18. The position is shown in FIG. 32. The distal end of the push rod is maintained in a position in which the IOL is largely projecting from the end of the ejection tube 44, but has not yet been fully pushed out of it. This allows the surgeon to place the IOL precisely without having to maintain pressure on the operating knob 34. When the desired position has been reached, the knob can be depressed farther to eject the IOL. At the same time, the latch pin moves distally beyond the end of the latch finger, and the latch finger swings to its relaxed condition, down from the position of FIG. 32, such that the latch pin 24 will slide rearward along and past the latch finger as the force on the operating knob is relieved, and the parts return to the position of FIG. 30. The ejection tube is removed from the corneal incision, and the parts can be returned to the position of FIG. 28, for removal of the empty cartridge 12. The instrument is ready for insertion of a new loaded cartridge.

While the preferred embodiment of the invention has been illustrated and described, it will be appreciated that various changes can be made therein without departing from the spirit and scope of the invention.

What is claimed is:

1. A device for inserting, controllably releasing and accurately positioning a folded intra-ocular lens into an eye comprising:
a plunger rod assembly in communication with an inserter housing, said inserter housing adapted to house an intra-ocular lens;
a first drive mechanism for providing contact between and causing lateral movement of said plunger rod assembly and said intra-ocular lens within said inserter housing; and
a latch pin and a latch finger configured for engagement with said latch pin for preventing proximal lateral movement of said plunger rod assembly so that said intra-ocular lens may be accurately positioned within said eye.

2. The device of claim 1, wherein said plunger rod assembly further comprises a push rod and a helical compression spring in biasing relation to said push rod.

3. The device of claim 2 wherein said plunger rod assembly further comprises a barrel within which said push rod is slideable, wherein said barrel has a proximal part and said push rod has a proximal part having a slide disc disposed thereon and said helical compression spring is slideable along said rod proximal part between said barrel proximal part and said slide disc.

4. The device of claim 3 further comprising a transversely projecting finger support secured to said barrel.

5. The device of claim 3, wherein said plunger rod assembly further comprises a knob having external threads rotatably secured to said push rod, wherein said barrel comprises inwardly projecting pins and wherein said external threads cooperate with said inwardly projecting pins.

6. The device of claim 1, wherein said plunger rod assembly further comprises a barrel within which said push rod is slideable, said barrel further comprising a slot and wherein said latch finger is carried on said barrel and centered over said slot and said latch pin is carried by said push rod.

7. The device of claim 6, wherein said plunger rod assembly further comprises a knob having external threads rotatably secured to said push rod, wherein said barrel comprises inwardly projecting pins and wherein said external threads cooperate with said inwardly projecting pins.

8. The device of claim 7 wherein the axial extent of said external threads along said knob is limited and said external threads thereby cooperate with said inwardly projecting pins over a limited distance.

9. The device of claim 6, wherein said latch finger comprises a ramp and a notched segment which is engageable with said latch pin.

10. The device of claims 1 further comprising a cartridge housed within said inserter housing, said cartridge having a folded intra-ocular lens positioned therein.

11. The device of claim 1 wherein said plunger rod assembly further comprises a barrel within which said push rod is slideable, wherein said barrel has a distal portion and wherein the device further comprises a cartridge having a folded intra-ocular lens positioned therein, wherein said cartridge is fitted into said distal portion of said barrel.

12. The device of claim 11 wherein said barrel has an interior bore and an exterior and said distal portion of said barrel has an axial slot communicating between said interior bore and said exterior and said bore allows said cartridge to be inserted downward into said bore of said barrel.

13. A device for inserting, controllably releasing and accurately positioning a folded intra-ocular lens into an eye comprising:
a barrel having a proximal portion, a distal portion having an axial slot, and an interior bore;
a push rod having a proximal part and a slide disc disposed thereon, said push rod slideable within said bore of said barrel;
a helical compression spring slideable along said proximal part of said rod between said proximal part of said barrel and said slide disc;
a cartridge having a folded intra-ocular lens positioned therein, wherein said cartridge is fitted into said axial slot on said distal portion of said barrel;
a first drive mechanism for providing contact between and causing lateral movement of said push rod and said intra-ocular lens within said lens cartridge; and
a latch pin and a latch finger configured for engagement with said latch pin for preventing proximal lateral movement of said push rod so that said intra-ocular lens may be accurately positioned within said eye.

14. The device of claim 13 further comprising a transversely projecting finger support secured to said barrel.

15. The device of claim 13, wherein said push rod further comprises a knob having external threads rotatably secured to said proximal part of said push rod, said barrel comprises inwardly projecting pins and wherein said external threads cooperate with said inwardly projecting pins.

16. The device of claim 15, wherein the axial extent of said external threads along said knob is limited and said external threads thereby cooperate with said inwardly projecting pins over a limited distance.

17. The device of claim 13, wherein said latch finger is carried on said barrel and centered over said slot and said latch pin is carried by said push rod.

18. The device of claim 13, wherein said latch finger comprises a ramp and a notched segment which is engageable with said latch pin.

19. A method of inserting an intra-ocular lens into an eye comprising:
providing an insertion device comprising a plunger rod assembly, an inserter housing and a control knob assembly;
providing a cartridge having a folded intra-ocular lens positioned therein;
loading said cartridge into said inserter housing;
actuating said control knob assembly to couple a distal end of said plunger rod assembly with said intra-ocular lens;
inserting a distal end of said cartridge into said eye;
applying a force to said control knob assembly to move said intra-ocular lens through said cartridge and partially eject said intra-ocular lens from said cartridge;
actuating a latch and pin mechanism of said plunger rod assembly and removing said force to said control knob assembly without causing movement of said plunger rod assembly;
positioning said intra-ocular lens in said eye;
applying a force to said control knob assembly to eject said intra-ocular lens into said eye; and
removing said distal end of said cartridge from said eye.

20. The method of claim 19 further comprising the step of biasing said plunger rod assembly against forward travel.

21. The method of claim 19 further comprising releasing said latch and pin mechanism of said plunger rod assembly.

22. A mechanism for releasably locking an intra-ocular lens insertion device comprising: a latch finger and a latch pin located on an inserter housing and a plunger rod assembly of said insertion device; wherein said latch finger and said latch pin are configured for releasable locking engagement to prevent involuntary retraction of said plunger rod assembly during use of said insertion device.

23. A method for controllably positioning and releasing an intra-ocular lens from an insertion device comprising:
applying pressure on said insertion device to advance said intra-ocular lens through said insertion device;
maintaining pressure on said insertion device until said intra-ocular lens projects from a distal end of said insertion device;
advancing a plunger rod assembly of said insertion device so that a leading edge of a latch pin on said plunger rod assembly engages an angled tip of a latch finger on a housing of said device, thereby locking said insertion device to prevent retraction of said intra-ocular lens;
releasing pressure on said insertion device;
positioning said intra-ocular lens in a patient's eye;
further advancing said plunger rod assembly so that said latch finger disengages from said latch pin, thereby unlocking said insertion device; and
releasing said intra-ocular lens into said patient's eye.
